(19) United States
(12) Patent Application Publication
(10) Pub. No.: US 2013/0189377 A1
(43) Pub. Date: Jul. 25, 2013

(54) COMPOSITIONS

(71) Applicants: Alasdair Cockett, Grays (GB); Paul Pay, Ashby de la Zouch (GB); Guy Cameron Nash, Guildford (GB)

(72) Inventors: Alasdair Cockett, Grays (GB); Paul Pay, Ashby de la Zouch (GB); Guy Cameron Nash, Guildford (GB)

(73) Assignee: Norgine BV, Amsterdam Zuid-Oost (NL)

(21) Appl. No.: 13/812,272

(22) PCT Filed: Jul. 26, 2011
(86) PCT No.: PCT/GB2011/001122

§ 371 Date: Mar. 26, 2013

(30) Foreign Application Priority Data
Jul. 27, 2010 (GB) 1012588.8
May 27, 2011 (GB) 1109121.2

(51) Int. Cl. A61K 33/06; A61K 33/04

(52) U.S. Cl. CPC A61K 33/06; A61K 33/04
USPC 424/682; 424/709; 264/109

(57) ABSTRACT

The present invention concerns solid compressed oral pharmaceutical compositions comprising a mixture of at least two sulphate salts selected from the group consisting of sodium, potassium and magnesium for use, in particular, in cleansing the colon or treating faecal impaction, constipation, faecal retention, intestinal gas and cramping, or flatulence in a mammal. Methods of producing such compositions are also disclosed.

The present invention relates to compositions for the treatment of orthostatic lavage, colon evacuation or colon cleansing, and also for the treatment of constipation, faecal impaction, faecal retention, intestinal gas and cramping, and flatulence. The invention relates in particular to compositions for colon cleansing. The invention further relates to methods of use of such compositions.

### GENERAL BACKGROUND

Colon cleansing is important prior to numerous diagnostic and surgical procedures, for example before colonoscopy, CT of colon, barium enema examination or colon surgery. It is also useful for preventing peritonitis or other complications after surgery on the lower intestine. Colon cleansing is also known as bowel cleansing and bowel lavage.

One method of colon cleansing is orthostatic intestinal lavage, in which a large volume of an electrolyte solution is ingested, either by drinking or by infusion through a nasogastric tube. Such lavage solutions are also known as bowel or gut lavage solutions. Consumption of the solution results in volume-induced diarrhea and thus cleansing of the colon. Most commonly used lavage solutions include polyethylene glycol, PEG, in addition to electrolytes. As an alternative, preparations containing phosphate salts have been proposed. One such commercially-available product is Fleet Phospho-Soda®. However, although such products are generally considered safe for most healthy adults, they may pose risks for adverse reactions in patients with renal, cardiac or hepatic diseases, and elderly patients in whom excess sodium absorption might be dangerous.

Many patients who take electrolyte-containing cleansing solutions find the taste of the solutions unpleasant and/or have difficulties taking the large volumes required. Those negative sensations lead to patient compliance being adversely affected.

Constipation is a widespread condition which generally gives rise to discomfort. The physical presence of faeces retained in the colon and/or the rectum gives rise to a feeling of malaise and headaches. In extreme cases of prolonged constipation dyschezia may result from the presence of scybala or faecaliths in the rectum.

Numerous treatments of constipation have been developed, including dietary manipulation (e.g. increasing the fibre content of the diet and removing foods considered to be constipation causing), laxatives and enemas. Laxatives are agents that promote and assist defecation. Osmotic laxatives act to retain water in the colonic lumen thereby counteracting the normal dehydrating action of the colon. By suppressing the dehydration action of the colon, the osmotic laxative produces a faecal stream which is softer, bulkier and easier to expel. Sodium sulphate (known as “Glauber's salt”) has been used as a laxative when diluted in a small volume of water and taken in small daily doses.

As for colon cleansing, many patients who take electrolyte-containing laxatives in powder, granular, solution, or in suspension form find the taste unpleasant and very salty. Those negative sensations lead to patient compliance being adversely affected.

Faecal impaction, also known as faecal retention, is the formation of a firm impassable mass of stool in the rectum or distal colon. Faecal impaction is often treated using methods similar to those described above for constipation and colon cleansing. A larger dose is generally needed than in the case of constipation treatment.

There remains a requirement for lavage preparations, laxatives and faecal impaction treatments which are safe to use and which are more acceptable to patients, leading to higher patient compliance.

### DESCRIPTION OF THE INVENTION

The invention provides a solid compressed oral pharmaceutical composition (such as a tablet) in unit dosage form which comprises a mixture of at least two, and preferably all three, salts selected from the group consisting of sodium sulphate, magnesium sulphate and potassium sulphate. Preferably, polyethylene glycol, if present, is present, in an amount of less than 50% by weight of the composition.

The compositions of the invention are suitable for use in, and may be used in, orthostatic lavage, colon evacuation or colon cleansing. They may also be used for the treatment of constipation, faecal impaction, faecal retention, intestinal gas and cramping, or flatulence.

In the compositions of the invention, the specified sulphate salt mixture preferably makes up at least 50%, e.g. at least 60%, for example at least 70, 80%, 90% or 95%, by weight of the composition, for example from 70 to 80% (such as 75%) or 80 to 95%. The remainder of the composition may be made up of one or more optional components selected for example from polyethylene glycol (PEG), other components having laxative activity, other electrolytes, excipients, sweeteners and flavouring agents. In one embodiment, no polyethylene glycol is present. If polyethylene glycol is present, it is preferably present in an amount of less than 50%, preferably less than 30%, preferably less than 20%, especially less than 10% by weight based on the weight of the composition.

The present inventors have also surprisingly found that in a solid compressed oral pharmaceutical composition in unit dosage form which comprises a mixture of at least two salts selected from the group consisting of sodium sulphate, magnesium sulphate and potassium sulphate that the absence of either sodium sulphate or magnesium sulphate adversely affects the tablet characteristics. However, the absence of either sodium sulphate or magnesium sulphate from the oral pharmaceutical composition can be compensated for by the addition of PEG. Pharmaceutical compositions according to the invention that comprise all three sulphates demonstrate acceptable characteristics and do not require the addition of PEG. PEG however may still be added to improve the performance of these compositions in use, i.e. in the treatment of orthostatic lavage, colon evacuation or colon cleansing, and also for the treatment of constipation, faecal impaction, faecal retention, intestinal gas and cramping, and flatulence.

Accordingly therefore, the present invention also provides a solid compressed oral pharmaceutical composition (such as a tablet) in unit dosage form which comprises a mixture of at least two salts selected from the group consisting of sodium sulphate, magnesium sulphate and potassium sulphate wherein if the composition is substantially free of either sodium sulphate or magnesium sulphate, the composition comprises PEG.

By the term “substantially free” we mean that the indicated sulphate, if present, constitutes such a low fraction of the total mass of the tablet that no discernible effect of its presence on the malleability of the tablet can be demonstrated.

The relative quantities of sodium, potassium and/or magnesium sulphate in the composition of the invention are preferably selected such that administration together with the directed quantity of water does not cause any significant electrolyte shifts in the patient. If the quantities of sodium, potassium and/or magnesium sulphate in the composition of the invention were dissolved in an appropriate amount of water, that solution would desirably be at least osmotic and possibly hyperosmotic. On ingestion, such a composition is preferably hypertonic or, once an appropriate quantity of additional liquid has been ingested thereafter, may be isotonic.

Suitably the composition of the invention contains two or, preferably, three of the following: 5 to 30 parts by weight of sodium sulphate; 1 to 5 parts by weight of potassium sulphate; 0.5 to 2.0 parts by weight of magnesium sulphate. Preferred are compositions containing two or, preferably, three of the following: 5 to 20 parts by weight of sodium sulphate; 1.5 to 3.0 parts by weight of potassium sulphate; 0.75 to 1.5 parts by weight of magnesium sulphate. Especially preferred are compositions containing two or, preferably, three of the following: 5 to 15 parts by weight sodium sulphate; 1.5 to 2.5 parts by weight of potassium sulphate; 0.8 to 1.2 parts by weight of magnesium sulphate. For example, one possible composition according to the invention comprises about 10 parts by weight of sodium sulphate; about 2 parts by weight of potassium sulphate; and about one part by weight of magnesium sulphate. In an alternative embodiment, particularly appropriate when PEG is present, the composition of the invention contains two or, preferably, three of the following: 2 to 40 parts by weight of sodium sulphate; 1 to 10 parts by weight of potassium sulphate; 2 to 20 parts by weight of magnesium sulphate (and optionally up to 50, for example 0.1 to 50, parts by weight of PEG). Preferred are compositions containing two or, preferably, three of the following: 10 to 30 parts by weight of sodium sulphate; 0.5-5 parts by weight of potassium sulphate; 10-30 parts by weight of magnesium sulphate (and optionally up to 45 parts, for example 25 to 45 parts, by weight of PEG); for example compositions containing two or, preferably, three of the following: 15 to 25 parts by weight of sodium sulphate; 1 to 4 parts by weight of potassium sulphate; 15-25 parts by weight of magnesium sulphate (and optionally up to 45 parts, for example 25 to 40 parts, by weight of PEG). For example, one possible composition according to the invention comprises about 20 parts by weight of sodium sulphate; about 3 parts by weight of potassium sulphate; and about 20 parts by weight of magnesium sulphate (and optionally about 35 parts by weight of PEG).

In a preferred embodiment, suitable for use in colon cleansing, sodium sulphate is present in each dosage unit in an amount such that a patient may conveniently take a course of treatment including from 20 to 50 g, more preferably 30 to 40 g, for example about 35 g. However, lower amounts may be used, such as in the order of 10, 1 or 0.1 g. A treatment may typically comprise two separate doses (perhaps taken one in the evening and one the following morning, see below), in which case the amount of sodium sulphate per dose may be from 10 to 25 g, more preferably 15 to 20 g, for example about 17.5 g.

In a preferred embodiment, suitable for use in colon cleansing, potassium sulphate is for example present in each dosage unit in an amount such that a patient may conveniently take a course of treatment including from 0.02 to 40 g, for example from 0.2 to 20 g, for example 1 to 10 g; for example about 6 g. A treatment may typically comprise two separate doses (perhaps taken one in the evening and one the following morning, see below), in which case the amount of potassium sulphate per dose may be from 0.01 to 20 g, for example from 0.1 to 10 g, for example 0.5 to 5 g, for example about 3 g.

In a preferred embodiment, suitable for colon cleansing, magnesium sulphate is for example present in each dosage unit in an amount such that a patient may conveniently take a course of treatment including from 0.02 to 80 g, for example from 0.1 to 40 g, for example 0.2 to 20 g, for example about 3 g. A treatment may typically comprise two separate doses (perhaps one taken in the evening and one the following morning), in which case the amount of magnesium sulphate per dose may be from 0.01 to 40 g, for example from 0.1 to 20 g, for example 0.1 to 10 g, for example about 1.5 g.

For the treatment of faecal impaction, suitable dosages of sulphate are typically a factor of around 4 lower than for colon cleansing, and the content of sulphate in the dosage units may be adjusted accordingly. For the treatment of constipation, intestinal gas and cramping, and flatulence, suitable dosages of sulphate are typically a factor of around 16 lower than for colon cleansing, and the content of sulphate in the dosage units may be adjusted accordingly. Particularly for the treatment of constipation, several doses may be taken per day.

Typically, the weight ranges given above for each sulphate component are based on anhydrous sulphate.

Any polyethylene glycol present in the compositions of the invention preferably has an average molecular weight of 2000 or greater. Preferably the PEG has an average molecular weight of 2500 or greater. Preferably the PEG has an average molecular weight of 4500 or lower. For example the PEG may be PEG 3350 or PEG 4000. PEG 3350 is most preferred. That product is available commercially, for example, from Dow Chemical Co., Clariant GmbH or BASF under the International Non-proprietary Name Macrogol 3350. When present, PEG, especially PEG 3350 or PEG 4000, comprises in the range of from 1 to 100 g, such as 12-14, 30-40 or 50-55 g, of the composition.

In use, the patient will generally take one or more dosage units of the compressed formulation, with and/or followed by appropriate amounts of water. Alternatively, the patient may be advised to dissolve or disperse the compressed pharmaceutical composition in water before taking the composition. In either event, the patient will typically be required to take from 60 to 1000 ml water, for example from 100 to 500 ml water, such as 200 to 330 ml water, with or immediately after ingesting the appropriate number of dosage units. This may then be followed by additional quantities over the next two to five, e.g. three, hours if required.

For example, for colon cleansing, it may be desirable to administer one dose in the evening and a second the following morning. Following each dose, a relatively large quantity of water is required, a typical total ingestion of water over the three hours following application of the composition of the invention being up to 4000, for example up to 3000, for example up to 2000, ml.

Treatment regimes using the compositions of the present invention are typically analogous to known treatment regimes for similar products in different forms. The total dosage of sulphate salts to be administered will depend upon the condition to be treated. For colon cleansing, a total treatment of from 200 to 400, for example from 250 to 350, especially from 295 to 325, mmol total sulphate, may be appropriate. This total amount is typically taken in two separate doses, for example one in the evening and one the following morning, of preferably about 100 to 200 (such as about 150 to 160) mmol total sulphates, but if required it may be taken in a single dose or as more than two doses.

For the treatment of faecal impaction, also known as faecal retention, dosages of about one quarter of those mentioned above may be used.

Paediatric treatments obviously require smaller dosages, typically half the adult dose. It is common to treat paediatric faecal impaction by taking a course of treatment over 7 days.

Particularly for colon cleansing, several dosage units, for example two, three or more dosage units, may be taken at the same time to make up an effective dose. Many dosage units may be required for high dosage treatments. For example, a full dosage may comprise, say, between 20 and 40 dosage units, for example 32 dosage units may be used for colon cleansing, with 8 used for faecal impaction and 2 for constipation.

In a preferred regime for cleansing the colon, the appropriate number of dosage units is taken by the patient, together with up to 4 litres of water. This may be done either by taking half of the total required number of dosage units with about 2 litres of water in the evening before a clinical examination or procedure, the remaining dosage units and about an additional 2 litres of water being taken in the early morning of the day of the examination or procedure. Alternatively, the whole treatment may be taken in the evening before the examination or procedure, or on the morning of the examination or procedure. Patients are told not to take any solid food from when they start to take the cleansing solution until after the examination or procedure.

Patients may be told to take the required amount of water over, say, one to two hours, for example by drinking a glassful every 10-15 minutes. Patients may be recommended to drink further quantities of water to prevent them from feeling very thirsty and becoming dehydrated.

A possible variant on the above regime is a regime in which the full treatment dose for colon cleansing for adult patients includes up to 4 litres of water taken orally prior to the colonoscopy or other examination or procedure in one of the following ways:

### 1) Split-Dose Regimen:

The evening before the colonoscopy or other examination or procedure, half the required number of dosage units of the composition of the invention are taken, the first litre (approximately 32 fluid ounces) of water is taken over one hour (one 250 ml/8 fluid ounce glass every 15 minutes), and then an additional litre of water is drunk. Then, on the morning of the colonoscopy or other examination or procedure, the remaining dosage units of the composition of the invention are taken, and a further litre of water is taken over one hour and then a final litre of water is drunk at least one hour prior to the start of the colonoscopy.

### 2) Evening-Only (Full-Dose) Regimen:

Around 6 pm in the evening before the colonoscopy or other examination or procedure, all the required dosage units according to the invention are taken together with a first litre of water over one hour (one 250 ml/8 fluid ounce glass every 15 minutes) and then about 1.5 hours later a second litre of water is taken over one hour. In addition, up to 2 litres of additional water are taken during the evening before the colonoscopy or other examination or procedure.

### 3) Evening-Only (Split-Dose) Regimen:

Around 6 pm in the evening before the colonoscopy or other examination or procedure, half the required dosage units according to the invention are taken together with a first litre of water over one hour (one 250 ml/8 fluid ounce glass every 15 minutes), and then about 1.5 hours later the remaining half of the required dosage units are taken together with a second litre of water taken over one hour. In addition, up to 2 litres of additional water are taken during the evening before the colonoscopy or other examination or procedure.

In all of these dosage regimes, instead of or in combination with water, a clear liquid such as soft drinks (e.g. ginger ale, cola, lemonade, orange), clear soup (e.g. chicken broth or beef bouillon), fruit juice (strained and without pulp, e.g. apple, orange, lemon, white grape), tea or coffee (without milk or dairy creamer) may be taken.

The compositions of the present invention may if desired contain salts (electrolytes) in addition to the specified mixture of sulphates, although in a preferred embodiment of the invention other salts comprise less than 20%, especially less than 10%, by weight of the composition. If additional salts are present, sodium, potassium and magnesium salts are preferred. Suitable salts include chlorides, bicarbonates, sorbates, benzoates, acetates, carbonates, citrates, fumarates, gluconates, malates, nitrates, succinatese or tartrates. Ascorbic acid or an ascorbate salt may be present.

If additional salts are present, chloride and bicarbonate salts are particularly preferred, for example sodium chloride, potassium chloride and/or sodium bicarbonate. Phosphate may be present if desired, but preferably no phosphate is present. If phosphate is present, it may be in the form of H₂PO₄ or (HPO₄)²⁻ ion, and it is preferably present in an amount of less than 20%, preferably less than 10%, by weight based on the weight of the composition. Preferably the composition of the invention contains insubstantial quantities, or no, salts other than the specified sulphate mixture.

The compositions of the invention will generally include one or more pharmaceutically acceptable excipients, in particular compression excipients. In general, the composition of the present invention should contain the minimum amount of compression excipients required to produce a coherent compressed unit dosage form. Suitable excipients include magnesium stearate, cellulose and derivatives of cellulose, starch and derivatives of starch, lactose and derivatives of lactose, dextrates, mannitols and sorbitols. Particularly in the case of an excipient selected from dextrates, mannitols and sorbitols, the compositions may have been spray dried during their manufacture. For example, suitable excipients include binders, for example cellulose, hydroxypropylcellulose, lactose, gelatine, polyvinylpyrrolidone, and starch; disintegrants, for example crosslinked polyvinylpyrrolidone, sodium starch glycolate, and crosslinked sodium carboxymethylcellulose (crosscarmellose sodium); glidants (flow aids), for example magnesium stearate, starch and talc; and fillers, for example cellulose, lactose, sucrose, glucose, manitol and sorbitol.

The compositions of the invention may contain insoluble excipients, but in one embodiment of the invention, no or only low levels of insoluble excipients are present. This may provide advantages in certain circumstances, for example, it avoids the possibility of an insoluble ingredient obscuring visualisation of colonic mucosa during colonoscopy. For example, the composition may be substantially free from insoluble cellulose, for example microcrystalline cellulose, or insoluble cellulose derivatives, for example microgranular sodium carboxymethylcellulose or crosscarmellose sodium; and/or insoluble mineral materials such as talc.

The compressed pharmaceutical compositions of the invention may if desired comprise components (other than or in addition to polyethylene glycol) with laxative activity, but preferably no such components are present. If present, additional laxatives may include osmotic laxatives, for example magnesium citrate, magnesium hydroxide, sorbitol or lactulose; or stimulant/irritant laxatives, for example bisacodyl or senna.

The compressed pharmaceutical compositions of the invention preferably comprise one or more sweeteners, or the composition may be substantially free from sweeteners. Artificial or naturally derived sweeteners may be used alone or in admixture. Suitable sweeteners include, but are not limited to, saccharin, sodium saccharin, sodium cyclamate, acesulfame potassium, thaumatin, neohesperidin dihydrochalcone, ammonium glycyrrhizinate and aspartame. The sweetener may make up between from about 0.01 to 1.0% by weight of the final composition, more preferably about 0.025 to 0.25% by weight, for example from 0.07 to 0.08% by weight. Where two sweeteners are used in admixture, the ratios between the two sweeteners may be in the range of about 1:10 to 10:1 by weight. Sweeteners made by chemically linking individual sweeteners together, e.g. “Twinsweet”®, may be used.

The compressed compositions of the invention preferably comprise one or more flavouring agents, or the composition may be substantially from flavouring agents. Suitable flavourings are available from a range of suppliers, for example International Flavors & Fragrances Inc. (IFF (GB) Ltd, Duddery Hill, Haverhill, Suffolk, CB9 8LG, United Kingdom) and include flavourings commonly used in foodstuffs and pharmaceuticals. Examples of suitable flavouring agents include Raspberry Lemon flavour, Lemon flavour, Lemon/Lime flavour, Cola flavour and Orange flavour. The quantity of flavouring needed to achieve a palatable taste depends on the identity of the flavouring agent. Typically, from 0.1 to 20% by weight of flavouring is appropriate. Preferably, 1.0% to 15% by weight of flavouring is used. The exact amount of flavouring that is appropriate depends, inter alia, on the intensity of the flavouring used. A flavour enhancer may be used in addition to a flavouring agent. Flavour enhancers include citric acid.

A salt masking agent may also be used. Examples of salt masking agents include sodium saccharide (E954), arabic gum/acacia (E414), aspartame (E951) and neospheridin dihydrochalcone (E0959) or mixtures thereof. The quantity of salt masking agent needed to achieve a palatable taste depends on the intensity of the agent. Typically, from 1 to 6% by weight of salt masking agent is appropriate. Preferably, from 2% to 5% by weight of salt masking agent is used, particularly 3 to 4% by weight. The exact amount of flavouring that is appropriate depends on the intensity of the salt masking agent used.

Preferred compositions according to the invention are those which comprise in addition to the specified salt mixture, an excipient of, for example magnesium stearate and/or microcrystalline cellulose, acesulfame potassium, flavouring, and a salt masker.

The compositions of the invention are for oral administration. Preferably they are in the form of tablets, pellets, pills, lozenges, caplets or capsules (for example gelatine capsules). In one embodiment, they may be chewable or suckable. Tablets and similar dosage units may be coated or uncoated. Coatings may be advantageous as they make larger tablets easier to swallow, and may mask unpleasant tastes. Suitable components of coating agents include, for example, cellulose derivatives (for example carboxymethyl cellulose, hydroxypropylmethyl cellulose or hydroxypropyl cellulose), gelatine, sugars, carrageenan and pectin.

Preferred dosage forms include lenticular tablets which can be round or oval. When the tablet is oval, the length of the tablet is for example from 5 to 30 mm, preferably from 10 to 30 mm, for example from 15 to 25 mm; the width is for example from 4 to 12 mm, preferably from 6 to 11 mm, for example from 8 to 10 mm; and the maximum thickness of the tablet is for example from 2 to 12 mm, preferably from 5 to 11 mm, for example from 8 to 10 mm.

When the tablet is round, the diameter of the tablet is for example 3 to 15 mm, preferably from 5 to 12 mm, more preferably from 7 to 10 mm, and the maximum thickness of the tablet is for example from 2 to 12 mm, preferably 5 to 11 mm, for example from 8 to 10 mm.

Capsules and other dosage forms may have similar dimensions to those given above for tablets.

Preferably, the weight of each dosage unit of the compressed oral composition is less than 5 g, preferably less than 3 g, especially less than 2 g. Preferably the weight of each dosage unit is more than 0.1 g, preferably more than 0.2 g, preferably more than 0.5 g, especially more than 1 g.

The invention also provides a solid compressed oral pharmaceutical composition according to the invention for the treatment of constipation, faecal impaction, faecal retention, intestinal gas and cramping, or flatulence; or for orthostatic lavage, colon evacuation or colon cleansing.

A solid compressed oral pharmaceutical composition, for example a tablet, is significantly more palatable than previously proposed liquid or dry powder or granular compositions, and is effective when administered. The compositions of the invention have the further advantage that they may be manufactured and packaged in a, convenient manner. For example, manufacturing and packaging techniques used in the manufacture of pharmaceutical tablets may be employed.

The solid compressed pharmaceutical compositions of the invention are typically made by mixing together the ingredients in dry powder form and compressing into the desired shape. Accordingly the invention also provides a method for preparing a composition according to the invention comprising the steps of:
(a) mixing together at least two, and preferably all three, salts selected from the group consisting of sodium sulphate, magnesium sulphate and potassium sulphate; optionally polyethylene glycol; and optionally one or more ingredients selected from: one or more sweeteners; one or more flavouring agents; one or more excipients; additional electrolytes; ascorbic acid and/or an ascorbate salt; and optional further components; and
(b) compressing the mixture into a solid compressed oral pharmaceutical composition in unit dosage form.

Methods of preparing compressed pharmaceutical compositions are well-known in the art, for example see Rudnic, E., and Schwartz, J. B. Oral Solid Dosage Forms, Chapter 92, Tablets, pp. 1615-1641, in Remington's, 19th Ed.

The invention also provides a method of cleansing the colon or treating faecal impaction, constipation, faecal retention, intestinal gas and cramping, or flatulence in a mammal, which comprises administering to the mammal an effective amount of a composition according to the invention. The preferred dosages administered in such a method are as given above.

The invention further provides a composition according to the invention for use in therapy, specifically for use in a method of cleansing the colon or treating faecal impaction, constipation, faecal retention, intestinal gas and cramping, or flatulence in a mammal.

The invention further provides the use of a mixture of two or three of sodium sulphate, potassium sulphate and magnesium sulphate for the manufacture of a medicament which is a compressed pharmaceutical composition in unit dosage form, for cleansing the colon. The invention also provides the use of a composition according to the invention for the manufacture of a medicament for the treatment of constipation, faecal impaction, faecal retention, intestinal gas and cramping, or flatulence in a mammal.

Preferably, the mammal referred to above is a human.

The following non-limiting Examples illustrate the invention.

### EXAMPLES

The quantities of ingredients in the following Examples represent desired treatment regimes. Bulk quantities of ingredients in the desired proportions are compressed into a plurality of tablets for use in colon cleansing using standard compression techniques. Preliminary granulation of one or more of the ingredients may be carried out if required. Customary excipients such as binders, disintegrants, flow agent, lubricants, flavourings and/or sweeteners are included as required.

The optimal number of tablets required to deliver the required dose will depend upon the intended usage.

Composition 1

| | |
|---|---|
| Sodium sulphate | 35 g |
| Potassium sulphate | 6.26 g |
| Magnesium sulphate | 3.2 g |

Typically 10 to 30 tablets are required to deliver this dosage.

Composition 2

| | |
|---|---|
| Sodium sulphate | 14.2 g |
| Magnesium sulphate | 12.0 g |

Typically 10 to 30 tablets are required to deliver this dosage.

Composition 3

| | |
|---|---|
| Sodium sulphate | 17.75 g |
| Potassium sulphate | 2.18 g |
| Magnesium sulphate | 15 g |

Typically 10 to 30 tablets are required to deliver this dosage.

Composition 4

| | |
|---|---|
| Sodium sulphate | 20.24 g |
| Potassium sulphate | 4.13 g |
| Magnesium sulphate | 17.1 g |

Typically 20 to 60 tablets are required to deliver this dosage.

Composition 5

| | |
|---|---|
| Sodium sulphate | 20.24 g |
| Potassium sulphate | 3.48 g |
| Magnesium sulphate | 17.1 g |
| PEG 3500 | 34 g |

Typically 40 to 120 tablets are required to deliver this dosage.

### Example 1

In this example, tablets with varying components and component weights were made as disclosed in Table 1 below.

TABLE 1

| | | | | Polyethylene | Total | |
|---|---|---|---|---|---|---|
| Com- | Sodium | Potassium | Magnesium | Glycol | Tablet | (Na):(K):(Mg):(PEG)* |
| position | Sulphate | Sulphate | Sulphate | 3350 | Weight | % w/w |
| 1 | 1.17 g | 0.21 g | 0.11 g | 0.00 g | 1.49 g | (78.5):(14.0):(7.4):(0.0) |
| 2 | 0.47 g | 0.40 g | 0.00 g | 0.00 g | 0.87 g | (54.0):(46.0):(0.0):(0.0) |
| 3 | 0.59 g | 0.07 g | 0.50 g | 0.00 g | 1.16 g | (50.8):(6.0):(43.1):(0.0) |
| 4 | 0.34 g | 0.07 g | 0.29 g | 0.00 g | 0.70 g | (48.6):(10.0):(41.4):(0.0) |
| 5 | 0.17 g | 0.03 g | 0.14 g | 0.28 g | 0.62 g | (27.4):(4.8):(22.6):(45.2) |
| 6 | 0.00 g | 0.47 g | 0.40 g | 0.00 g | 0.87 g | (0.0):(54.0):(46.0):(0.0) |
| 7 | 0.51 g | 0.09 g | 0.42 g | 0.00 g | 1.02 g | (50.0):(8.8):(41.1):(0.0) |
| 8 | 0.47 g | 0.00 g | 0.40 g | 0.00 g | 0.87 g | (54.0):(0.0):(46.0):(0.0) |
| 9 | 0.47 g | 0.40 g | 0.00 g | 0.28 g | 1.15 g | (40.7):(34.8):(0.0):(24.3) |
| 10 | 0.00 g | 0.47 g | 0.40 g | 0.28 g | 1.15 g | (0.0):(40.9):(34.8):(24.3) |
| 11 | 0.00 g | 0.40 g | 0.47 g | 0.28 g | 1.15 g | (0.0):(34.8):(40.9):(24.3) |

*refers to the respective % w/w of the various components of the tablet in the order: Sodium Sulphate-Potassium Sulphate-Magnesium Sulphate-Polyethylene glycol.

For each tablet composition, the relevant component was weighed and transferred to a pestle and mortar. Once all of the components of the composition had been added to the mortar, the resulting powder composition was milled to a uniform consistency. The powder was then transferred to a KBR die (16 mm diameter). The Die and press were set up as per manual (SPECAC Manually Operated Hydraulic Press)

The powder was pressed to 10 metric tonnes and held at that pressure for 30 seconds. The resulting tablet was removed, visually inspected and tested for malleability.

### Results.

Each composition compressed into a tablet of varying thickness. Compositions 1 and 2 showed signs of crumbling on the edges once removed from the die.

Compositions 5, 6, 7, 8, 9, 10, 11 had a shiny surface whereas compositions 1 to 4 had a chalky surface.

Each tablet was dropped from a height ranging from 10 cm to 25 cm onto a hard surface. Compositions 1, 3, 4, 5, 8, 9, 10 and 11 did not break. Compositions 2 broke at a height of 15 cm, compositions 6 broke at a height of 12 cm.

### CONCLUSIONS

Tablets 2 and 6 are double sulphate tablets, without added PEG, where magnesium sulphate and sodium sulphate are, respectively absent. These tablets broke when dropped at a height of 15 cm and 12 cm respectively. When PEG was added (tablets 9 and 10), the tablets did not break when dropped from a height of 25 cm. On the other hand, a triple sulphate tablet, without added PEG, also did not break. A double sulphate tablet of magnesium and sodium sulphate, tablet 8, without additional PEG, demonstrated acceptable characteristics.

These results demonstrate that magnesium and sodium sulphate are important components for an acceptable multi-sulphate tablet having at least two different sulphate salts. The absence of either of these components adversely affects tablet characteristics. That absence however, can be compensated for by the inclusion of PEG. A tablet comprising all three sulphates however, demonstrated acceptable characteristics without the addition of PEG. The addition of PEG to a three sulphate tablet did not adversely affect it.

1-23. (canceled)

24. A solid compressed oral pharmaceutical composition in unit dosage form which comprises a mixture of at least two salts selected from the group consisting of sodium sulphate, magnesium sulphate and potassium sulphate.

25. A composition of claim 1 which comprises a mixture of all three sulphates.

26. A composition as claimed in claim 1, which further comprises less than 50% by weight of polyethylene glycol based on the weight of the composition.

27. A composition as claimed in claim 26, which further comprises less than 30% by weight of polyethylene glycol based on the weight of the composition.

28. A composition as claimed in claim 1, which is substantially free of polyethylene glycol.

29. A composition as claimed in claim 1, in which said mixture of sulphates makes up at least 50% by weight of the composition.

30. A composition as claimed in claim 29, in which said mixture of sulphates makes up at least 60% by weight of the composition.

31. A composition as claimed in claim 30, in which said mixture of sulphates makes up from 70 to 80% or 80 to 95% by weight of the composition.

32. A composition as claimed in claim 31, in which said mixture of sulphates makes up 75% by weight of the composition.

33. A composition as claimed in claim 1, which comprises two or three of the following: 5 to 30 parts by weight of sodium sulphate; 1 to 5 parts by weight of potassium sulphate; and 0.5 to 2.0 parts by weight of magnesium sulphate.

34. A composition as claimed in claim 33, which comprises two or three of the following: 5 to 15 parts by weight sodium sulphate; 1.5 to 2.5 parts by weight of potassium sulphate; and 0.8 to 1.2 parts by weight of magnesium sulphate.

35. A composition as claimed in claim 34, which comprises about 10 parts by weight of sodium sulphate; about 2 parts by weight of potassium sulphate; and about one part by weight of magnesium sulphate.

36. A composition as claimed in claim 1, which comprises two or three of the following: 2 to 40 parts by weight of sodium sulphate; 1 to 10 parts by weight of potassium sulphate; and 2 to 20 parts by weight of magnesium sulphate; and optionally up to 50 parts by weight of polyethylene glycol.

37. A composition as claimed in claim 36, which comprises compositions containing two or three of the following: 15 to 25 parts by weight of sodium sulphate; 1 to 4 parts by weight of potassium sulphate; and 15 to 25 parts by weight of magnesium sulphate; and optionally 25 to 40 parts by weight of polyethylene glycol.

38. A composition according to claim 36 which comprises about 13 parts of sodium sulphate, about 2.5 parts of potassium sulphate, about 11 parts of magnesium sulphate and about 21 parts of polyethylene glycol.

39. A composition according to claim 1, which comprises a mixture of at least two salts selected from the group consisting of sodium sulphate, magnesium sulphate and potassium sulphate wherein if the composition is substantially free of either sodium sulphate or magnesium sulphate then the composition comprises polyethylene glycol.

40. A composition as claimed in claim 1, which also comprises one or more pharmaceutically acceptable excipients selected from binders, disintegrants, glidants and fillers.

41. A method for preparing a composition according to claim 1, comprising the steps of:
(a) mixing together at least two salts selected from the group consisting of sodium sulphate, magnesium sulphate and potassium sulphate; and
(b) compressing the mixture into a solid compressed oral pharmaceutical composition in unit dosage form.

42. A method of cleansing the colon or treating faecal impaction, constipation, faecal retention, intestinal gas and cramping, or flatulence in a mammal, which comprises orally administering to the mammal an effective amount of a composition as claimed in claim 1.

43. A method as claimed in claim 42, which is a method of cleansing the colon, and in which said dosage units are such that there is administered to the patient a dosage of two or three of the following: 20 to 50 g sodium sulphate; 0.02 to 40 g potassium sulphate; 0.01 to 80 g magnesium sulphate.

44. A method as claimed in claim 43, in which said dosage units are such that there is administered to the patient a dosage of two or three of the following: about 35 g sodium sulphate; about 6 g potassium sulphate; about 3 g magnesium sulphate.

45. A method as claimed in claim 42, which also comprises the ingestion of up to 4000 ml of water or clear liquid.

46. A method as claimed in claim 42, which is a method of treating faecal impaction, and in which said dosage units are such that there is administered to the patient a dosage of two or three of the following: 5 to 12.5 g sodium sulphate; 0.005 to 10 g potassium sulphate; 0.005 to 20 g magnesium sulphate.

47. A method as claimed in claim 42, which is a method of treating constipation, and in which said dosage units are such that there is administered to the patient a dosage of two or three of the following: 1.25 to 3.125 g sodium sulphate; 0.00125 to 2.5 g potassium sulphate; 0.00125 to 5 g magnesium sulphate.
